(19) United States
(12) Patent Application Publication
(10) Pub. No.: US 2026/0200815 A1
(43) Pub. Date: Jul. 16, 2026

(54) PROCESS FOR CATALYTIC CRACKING OF LIGHT HYDROCARBONS TO REDUCE ACETYLENE FORMATION

(71) Applicant: Chevron U.S.A. Inc., San Ramon, CA (US)

(72) Inventors: Xiaoying Ouyang, El Cerrito, CA (US); Howard Steven Lacheen, Richmond, CA (US); Christopher Declan Lane, Kensington, CA (US); Minhaal Hussein Kalyan, Torrance, CA (US); Steve Brogden, Carmel Valley, CA (US); Huping Luo, Moraga, CA (US)

(21) Appl. No.: 19/021,741

(22) Filed: Jan. 15, 2025

(51) Int. Cl. C07C 4/06

(52) U.S. Cl. CPC C07C 4/06

(57) ABSTRACT

A non-oxidative conversion process includes processing a light hydrocarbon feed stream comprising C₁ to C₃ alkanes in a reactor under catalytic cracking conditions including a temperature of about 850° C. and up to 1020° C. and a residence time of from about 1 second to about 15 seconds in the presence of a catalytic cracking catalyst, thereby producing a product effluent stream comprising a C₂ to C₁₀ hydrocarbon product and hydrogen. The product effluent stream contains less than or equal to about 6.5 wt. % of acetylene.

### BACKGROUND

The ongoing search for alternatives to crude is increasingly driven by a number of factors. These include diminishing petroleum reserves, higher anticipated energy demands, and heightened concerns over greenhouse gas emissions from sources of non-renewable carbon. In view of its abundance in natural gas reserves, as well as in gas streams obtained from biological sources (biogas), natural gas has become the focus of a number of possible routes for providing liquid hydrocarbons. Natural gas occurs underground and is present as a gas when it comes out of the ground. Natural gas primarily consists of methane (CH₄), and additionally some other hydrocarbons such as ethane (C₂H₆) and propane (C₃H₈). Accordingly, converting light hydrocarbons such as methane to high value products such as hydrogen, olefins and aromatics has become an attractive option.

### SUMMARY

In accordance with an illustrative embodiment, a non-oxidative conversion process comprises:
processing a light hydrocarbon feed stream comprising C₁ to C₃ alkanes in a reactor under catalytic cracking conditions comprising a temperature of about 850° C. and up to 1020° C. and a residence time of from about 1 second to about 15 seconds in the presence of a catalytic cracking catalyst, thereby producing a product effluent stream comprising a C₂ to C₁₀ hydrocarbon product and hydrogen,
wherein the product effluent stream contains less than or equal to about 6.5 wt. % of acetylene.

### BRIEF DESCRIPTION OF THE DRAWINGS

In combination with the accompanying drawings and with reference to the following detailed description, the features, advantages, and other aspects of the implementations of the present disclosure will become more apparent, and several implementations of the present disclosure are illustrated herein by way of example but not limitation. The principles illustrated in the example embodiments of the drawings can be applied to alternate processes and apparatus. Additionally, the elements and features shown in the drawings are not necessarily to scale, emphasis instead being placed upon clearly illustrating the principles of the example embodiments. Certain dimensions or positions may be exaggerated to help visually convey such principles. In the drawings, the same reference numerals used in different embodiments designate like or corresponding, but not necessarily identical, elements. In the accompanying drawings:

FIG. 1 is a bar chart illustrating the yields versus residence time for the reaction of Example 1, according to an illustrative embodiment.

FIG. 2 is a bar chart illustrating the yields versus residence time for the reaction of Example 2, according to an illustrative embodiment.

### DETAILED DESCRIPTION

Various illustrative embodiments described herein are directed to reactor systems and processes for non-oxidative converting of light hydrocarbons into hydrogen and other valuable products with little to no acetylene formation.

Direct conversion of light hydrocarbons such as methane (CH₄), ethane and propane under non-oxidative conditions can produce higher molecular weight hydrocarbons, such as olefins, alkynes and aromatics (e.g., benzene), as value-added chemicals and at the same time produce hydrogen that can be used to make, for example, clean and zero carbon fuel. Hydrogen is one of the more important options for future clean energy. In addition, among the other valuable products, ethylene and benzene hold significant industrial value due to their extensive applications. Acetylene is also a useful product as it can serve, for example, as an essential feedstock for various chemical syntheses, including the production of vinyl chloride, acrylic acid, and other important intermediates. Furthermore, acetylene finds utility as a fuel for oxyacetylene welding and cutting.

However, despite its industrial significance, the production of acetylene during the catalytic cracking of light hydrocarbons presents several challenges. One problem is that acetylene's high flammability poses serious safety risks in handling and processing. Another problem is that excessive acetylene production can lead to operational inefficiencies and increased downstream separation costs. Conventional methods to mitigate excessive acetylene formation rely on selective hydrogenation, often employing precious metal catalysts to reduce acetylene levels while preserving the desired olefins. These methods, however, introduce additional operational complexity and cost.

Non-oxidative catalytic cracking of light hydrocarbons, particularly natural gas, offers a more straightforward and cost-effective process compared to traditional naphtha cracking or ethylene cracking routes. This approach eliminates the need for steam cracking, simplifying feedstock preparation and reducing operational costs. However, the high temperatures required for non-oxidative cracking can exacerbate acetylene formation, making it essential to develop methods for suppressing its production without compromising overall conversion efficiency or product distribution.

In view of these challenges, there is a need for solutions to handle acetylene produced during the conversion process to reduce the associated safety risk as well as simplify the overall process. The non-limiting illustrative embodiments described herein overcome the foregoing drawbacks by providing an approach to achieve this objective by controlling reaction parameters to reduce the formation of acetylene.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology can be applied, as long as that definition does not conflict with any other disclosure or definition applied herein or render indefinite or non-enabled any claim to which that definition is applied. To the extent that any definition or usage provided by any document incorporated herein by reference conflicts with the definition or usage provided herein, the definition or usage provided herein controls.

While systems and processes are described in terms of “comprising” various components or steps, the systems and processes can also “consist essentially of” or “consist of” the various components or steps, unless stated otherwise.

The terms “a,” “an,” and “the” are intended to include plural alternatives, e.g., at least one. The terms “including,” “with,” and “having,” as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means ±20% of the stated value, ±15% of the stated value, ±10% of the stated value, ±5% of the stated value, ±3% of the stated value, or ±1% of the stated value.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

The term “continuous” as used herein shall be understood to mean a system that operates without interruption or cessation for a period of time, such as where reactant(s) and catalyst(s) are continually fed into a reaction zone and products are continually or regularly withdrawn without stopping the reaction in the reaction zone.

A “fresh catalyst” as used herein denotes a catalyst which has not previously been used in a catalytic process.

A “spent catalyst” as used herein denotes a catalyst that has less activity at the same catalytic cracking conditions (e.g., temperature, pressure, inlet flows) than the catalyst had when it was originally exposed to the process. This can be due to a number of reasons, several non-limiting examples of causes of catalyst deactivation are coking or carbonaceous material sorption or accumulation, steam or hydrothermal deactivation, metals (and ash) sorption or accumulation, attrition, morphological changes including changes in pore sizes, cation or anion substitution, and/or chemical or compositional changes.

A “regenerated catalyst” as used herein denotes a catalyst that had become spent, as defined above, and was then subjected to a process that increased its activity to a level greater than it had as a spent catalyst. This may involve, for example, reversing transformations or removing contaminants outlined above as possible causes of reduced activity. The regenerated catalyst typically has an activity that is equal to or less than the fresh catalyst activity.

The term “zone” can refer to an area including one or more equipment items and/or one or more sub-zones. Equipment items can include one or more reactors or reactor vessels, absorption units, separation vessels, distillation towers, heaters, heat exchangers, pipes, pumps, compressors, and controllers. Additionally, an equipment item, such as a reactor, dryer, or vessel, can further include one or more zones or sub-zones.

The term “effluent” refers to a stream that is passed out of a reactor, a reaction zone, or an absorption unit following a particular reaction or separation. Generally, an effluent has a different composition than the stream that entered the reactor, reaction zone, or absorption unit. It should be understood that when an effluent is passed to another component or system, only a portion of that effluent may be passed. For example, a slipstream may carry some of the effluent away, meaning that only a portion of the effluent may enter the downstream component or system.

The term “primarily” shall be understood to mean an amount greater than 50%, e.g., 50.01 to 100%, or any range between, e.g., 51% to 95%, 75% to 90%, at least 60%, at least 70%, at least 80%, etc.

Illustrative embodiments address the above and other issues with existing processes for converting a light hydrocarbon stream into a product effluent comprising hydrogen and a C₂ to C₁₀ hydrocarbon product such as ethylene and benzene with little to no acetylene formation. The system and methodology provide many advantages, examples of which are mentioned herein. For example, the non-limiting illustrative embodiments described herein overcome the drawbacks discussed above by providing non-oxidative conversion processes to minimize acetylene formation from catalytic cracking a light hydrocarbon feed stream. In particular, the non-limiting illustrative embodiments described herein provide non-oxidative conversion processes demonstrating that by lowering the reaction temperature to 1020° C. or lower while increasing the residence time, it is possible to achieve equivalent conversion levels with significantly lower acetylene selectivity as compared to a reaction temperature greater than 1020° C. and short residence times. This balance advantageously increases the production of ethylene and benzene, which are more desirable products for industrial applications. In addition to lower temperatures and longer residence times, if the pressure can be increased the acetylene formation can be further suppressed. Therefore, a combination of lower temperatures, longer residence times, and higher pressures can minimize acetylene formation during the catalytic cracking of light hydrocarbons.

### Light Hydrocarbon Feed Stream

The light hydrocarbon feed stream to be employed is not particularly limited and may include, for example, C₁ to C₆ or C₁ to C₄ or C₁ to C₃ or C₁ to C₂ alkanes such as methane, ethane, or natural gas either pure or in any suitable mixture. In some embodiments, the light hydrocarbon feed stream may also contain minor amounts of other components including, for example, carbon dioxide, sulfur compounds such as H₂S, water, nitrogen, and mixtures thereof. In some embodiments the light hydrocarbon feed stream may also include steam, superheated steam, an inert gas such as nitrogen, or any mixture thereof. In some embodiments, the light hydrocarbon feed stream to be employed may include any suitable composition such that the resulting product includes at least hydrogen.

In some embodiments, the light hydrocarbon feed stream comprises methane or natural gas such as, for example, a light hydrocarbon feed stream comprising greater than about 80%, or greater than about 90%, or greater than about 95%, or greater than about 99% methane. As used herein, natural gas comprises methane and potentially higher alkanes, carbon dioxide, nitrogen or other gases, and/or sulfur-containing compounds such as hydrogen sulfide, and mixtures thereof. In illustrative embodiments, the light hydrocarbon feed stream may further contain a portion of the produced products that are recycled back to the light hydrocarbon feed stream along with unreacted methane.

### Catalytic Cracking Catalyst

There is no particular limitation on the type of catalytic cracking catalyst to be employed in the catalytic cracking reaction described herein. In an illustrative embodiment, as may be combined with one or more of the preceding paragraphs, the catalytic cracking catalyst can correspond to one or more catalytically active metals in particulate form and/or supported on particles. Catalytically active metals for use in the catalytic cracking process can include those from Groups 4-12 of the IUPAC Periodic Table of Elements. Suitable metals include, for example, iron, nickel, molybdenum, zinc, vanadium, tungsten, cobalt, ruthenium, and any combination thereof. The catalytically active metal may be present as a solid particulate in elemental form or as an organic compound or an inorganic compound such as a sulfide or other ionic compound. Metal or metal compound nanoaggregates may also be used to form the solid particulates.

A catalyst in the form of a solid particulate is generally a compound of a catalytically active metal, or a metal in elemental form, either alone or supported on a refractory material such as an inorganic metal oxide (e.g., alumina, silica, titania, zirconia, and any combination thereof). Other suitable refractory materials can include carbon, coal, and clays. Zeolites and non-zeolitic molecular sieves are also useful as solid supports. Generally, a supported catalyst can have from about 0.01 to about 30 wt. % of the catalytic active metal based on the total weight of the catalyst.

A catalyst bed/zone may have a mixture of two types of catalysts and/or successive beds/zones, including stacked beds, and may have the same or different catalysts and/or catalyst mixtures.

The catalytic cracking catalyst may be in any of the commonly used catalyst shapes such as, for example, spheres, granules, pellets, chips, rings, extrudates, or powders that are well-known in the art.

In some embodiments, the catalytic cracking catalyst can be one or more of a fresh catalyst, regenerated catalyst and spent catalyst.

### Catalytic Cracking Reaction

Catalytic cracking of light hydrocarbons, particularly methane, is a free-radical chain reaction that progresses through a sequential mechanism. Using methane as an example, the reaction begins with the formation of ethane, which undergoes further cracking to form ethylene, acetylene, benzene, polynuclear aromatics, and eventually coke. The extent of the reaction is influenced by several parameters, which may serve as control knobs for directing the selectivity of the reaction.

For example, initiation, propagation, and termination are the three key types of steps in free-radical chain reactions. In the initiation step, the high-temperature environment for catalytic cracking initiates the breaking of C—H bonds in methane, generating methyl radicals. During the propagation step, the methyl radicals react to form ethane, which subsequently cracks into ethylene and acetylene through a series of radical-mediated steps. The reaction can either terminate at intermediate products like ethylene and benzene or progress to form acetylene and polynuclear aromatics, depending on the reaction conditions.

Without being bound by theory, it is believed that the key to minimizing acetylene production lies in controlling the depth of the chain reaction. The illustrative embodiments described herein identify the following parameters as critical control points: temperature, residence time and pressure. High temperatures accelerate the reaction kinetics, increasing conversion efficiency but also promote acetylene formation, as acetylene formation is more favorable thermodynamically than benzene. Thus, it has been found that lowering the temperature can thermodynamically suppress the formation of acetylene. However, it has also been found that lowering the temperature alone without changing other parameters will decrease the conversion. Short residence times favor the formation of lighter products such as ethylene and acetylene while suppressing benzene formation. It has also been found that extending the residence time allows for secondary reactions that convert acetylene into benzene and other heavier products, thereby reducing acetylene selectivity. As shown in Table 1, higher pressures favor the formation of products such as ethylene and benzene that lead to lower volume expansion, while suppressing acetylene and coke formation which have high volume expansion.

TABLE 1

| | | Suppression by |
|---|---|---|
| Formula | Volume Expansion | Pressure |
| CH₄ → 0.5 C₂H₄ + H₂ | 1.5 | Low |
| CH₄ → 0.5 C₂H₂ + 1.5 H₂ | 2 | High |
| CH₄ → 0.167 C₆H₆ + 1.5 H₂ | 1.667 | Medium |
| CH₄ → C + 2 H₂ | 2 | High |

Applicant has surprisingly found that changing a single parameter does not achieve the desired lower acetylene selectivity. Moreover, different combinations of parameter changes can result in significantly different results. For example, higher temperature and longer residence time increase conversion, decrease any light products such as acetylene, and increase coke formation. Secondly, higher temperature and shorter residence time increase light products such as acetylene and decrease benzene while still maintaining a relatively high conversion.

The present disclosure demonstrates that by lowering the reaction temperature while increasing the residence time, comparable conversion levels can be achieved with significantly reduced acetylene selectivity compared to operating at high temperature and short residence time. This approach improves the production of ethylene and benzene, which are more desirable products for industrial applications. Additionally, increasing the pressure further suppresses acetylene formation. Therefore, a combination of lower temperature, longer residence time, and higher pressure may provide an effective strategy for minimizing acetylene formation during the catalytic cracking of light hydrocarbons.

The non-limiting illustrative embodiments described herein are directed to a process for non-oxidative conversion of a light hydrocarbon feed comprising primarily C₁-C₃ alkanes to a C₂ to C₁₀ hydrocarbon product and hydrogen with less than or equal to 6.5 wt. % acetylene by processing a light hydrocarbon feed stream comprising C₁ to C₃ alkanes in a reactor under catalytic cracking conditions described below in the presence of a catalytic cracking catalyst. Suitable reactors include, for example, stacked bed reactors, fixed bed reactors, ebullating bed reactors, continuous stirred tank reactors, fluidized bed reactors, spray reactors, liquid/liquid contactors, slurry reactors, slurry bubble column reactors, liquid recirculation reactors, and combinations thereof. In aspects, the reactor can have a power source configured to supply energy (e.g., electricity, combustible material) to heat the reaction zone of the reactor where catalytic cracking takes place.

In non-limiting illustrative embodiments, the process involves processing the light hydrocarbon feed stream comprising C₁ to C₃ alkanes in the presence of a catalytic cracking catalyst in a reactor under catalytic cracking conditions comprising a temperature of about 850° C. and up to 1020° C. and a residence time of from about 1 second to about 15 seconds thereby producing a product effluent stream comprising a C₂ to C₁₀ hydrocarbon product and hydrogen, wherein the product effluent stream contains less than or equal to about 6.5 wt. % of acetylene.

In some embodiments, suitable catalytic cracking conditions include a temperature ranging from about 850° C., or from about 920° C., or from about 950° C., or from about 970° C., and up to about 1020° C. or up to about 1000° C. In some embodiments, suitable catalytic cracking conditions further include a residence time of about 1 second, or about 3 seconds, or about 5.5 seconds, or about 7.5 seconds, and up to about 15 seconds, or up to about 10 seconds. Any of the lower limits described above can be combined with any of the upper limits.

In some embodiments, suitable catalytic cracking conditions include a temperature of about 850° C. and up to 1000° C. and a residence time of from about 5.5 second to about 10 seconds. In some embodiments, suitable catalytic cracking conditions include a temperature of about 920° C. and up to 1000° C. and a residence time of from about 5.5 second to about 10 seconds. In some embodiments, suitable catalytic cracking conditions include a temperature of about 970° C. and up to 1000° C. and a residence time of from about 1 second to about 7.5 seconds. In some embodiments, suitable catalytic cracking conditions include a temperature of about 920° C. and up to 970° C. and a residence time of from about 7.5 second to about 15 seconds.

In some embodiments, suitable catalytic cracking conditions include a temperature of about 850° C. and up to 1000° C. and a residence time of from about 5.5 second to about 10 seconds, and the product effluent stream contains no amount of acetylene. In some embodiments, suitable catalytic cracking conditions include a temperature of about 920° C. and up to 1000° C. and a residence time of from about 5.5 second to about 10 seconds, and the product effluent stream contains no amount of acetylene.

In some embodiments, suitable catalytic cracking conditions further include a pressure ranging from ambient (i.e., 0 atmosphere (atm)), or about 2 atm, and up to about 5 atm. It is believed that higher pressures can increase the formation of products, such as ethylene and benzene, that lead to low volume expansion, while suppressing acetylene and coke formation which have high volume expansion.

The produced product effluent stream derived from the light hydrocarbon feed stream typically comprises a C₂ to C₁₀ hydrocarbon product and hydrogen. By employing the particular catalytic cracking conditions discussed below during the process, the C₂ to C₁₀ hydrocarbon product produced during the reaction will limit the amount of acetylene to a level of less than about 6.5 wt. %. In some embodiments, the C₂ to C₁₀ hydrocarbon product produced during the reaction will limit the amount of acetylene to a level of less than about 6 wt. %. In some embodiments, the C₂ to C₁₀ hydrocarbon product produced during the reaction will limit the amount of acetylene to a level of less than about 5 wt. %. In some embodiments, the C₂ to C₁₀ hydrocarbon product produced during the reaction will limit the amount of acetylene to a level of less than about 4 wt. %. In some embodiments, the C₂ to C₁₀ hydrocarbon product produced during the reaction will limit the amount of acetylene to a level of less than about 3.8 wt. %. In some embodiments, the C₂ to C₁₀ hydrocarbon product produced during the reaction will limit the amount of acetylene to a level of less than about 3.5 wt. %. In some embodiments, the C₂ to C₁₀ hydrocarbon product produced during the reaction will limit the amount of acetylene to a level of less than about 3 wt. %. In some embodiments, the C₂ to C₁₀ hydrocarbon product produced during the reaction will limit the amount of acetylene to a level of less than about 2 wt. %. In some embodiments, the C₂ to C₁₀ hydrocarbon product produced during the reaction will limit the amount of acetylene to a level of less than about 1 wt. %. In some embodiments, the C₂ to C₁₀ hydrocarbon product produced during the reaction will limit the amount of acetylene to a level of less than about 0.5 wt. %. In some embodiments, the C₂ to C₁₀ hydrocarbon product produced during the reaction will limit the amount of acetylene to a level of less than about 0.1 wt. %. In some embodiments, the C₂ to C₁₀ hydrocarbon product produced during the reaction will produce no acetylene.

The C₂ to C₁₀ hydrocarbon product can be, for example, saturated, unsaturated, aromatic, or a mixture of such compounds. Examples of aromatic hydrocarbons include benzene, toluene, xylene, naphthalene, and methylnaphthalene. In some embodiments the C₂ to C₁₀ hydrocarbon product may comprise ethylene, propylene, benzene, naphthalene, and various mixtures thereof depending upon the desired products and reactions used. In addition, as one skilled in the art will readily appreciate, the resulting C₂ to C₁₀ hydrocarbon product can be one of a liquid hydrocarbon product, a gaseous hydrocarbon product, a solid hydrocarbon product and combinations thereof depending on the particular methane conversion process.

In some embodiments the C₂ to C₁₀ hydrocarbon product may comprise from about 1 wt. % to about 25 wt. % ethylene and from about 1 wt. % to about 30 wt. % benzene. In some embodiments the C₂ to C₁₀ hydrocarbon product may comprise from about 5 wt. % to about 15 wt. % ethylene and from about 5 wt. % to about 18 wt. % benzene.

Following catalytic cracking, the hydrogen can be separated from the C₂ to C₁₀ hydrocarbon product and sent for further processing. The C₂ to C₁₀ hydrocarbon product can be sent to a fractionator where the various components can be separated from each other. For example, the process may further include fractionating the aromatics stream to produce one or more of a benzene stream, a toluene stream, or a mixed xylenes stream. The benzene from the benzene stream may be catalytically hydrogenated to produce cyclohexane, for example, using a hydrogenation unit.

The following non-limiting examples are illustrative of the present disclosure.

### Example 1

In this example, the reaction involved the catalytic cracking of a simulated natural gas mixture composed of 90% methane, 4% ethane, 4% propane, and 2% carbon dioxide in the presence of a silicon dioxide-based catalyst. The reaction was carried out at a reaction temperature of 950° C. and at ambient pressure (1 atm) across a range of residence times, from 0.37 seconds to 5.46 seconds.

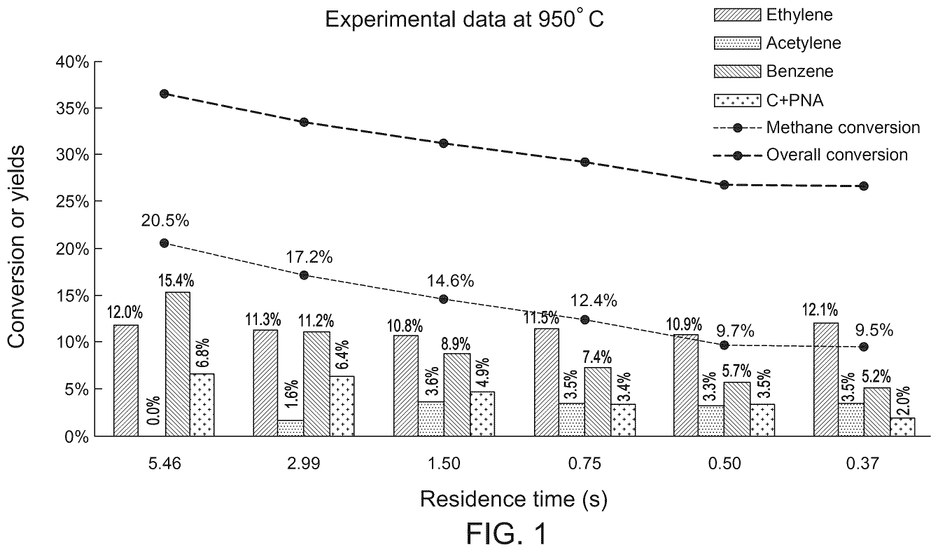

FIG. 1 shows a bar chart illustrating the results by comparing the methane conversion and overall conversion with the selectivity for ethylene, acetylene, benzene, and heavier products including coke (C) and polynuclear aromatics (PNA). The data shows the impact of residence time on product distribution and conversion during the catalytic cracking process. As can be seen in FIG. 1, the acetylene formation was below the detectable limit at a residence time of 5.46 seconds, while achieving both ethylene and benzene selectivity.

### Example 2

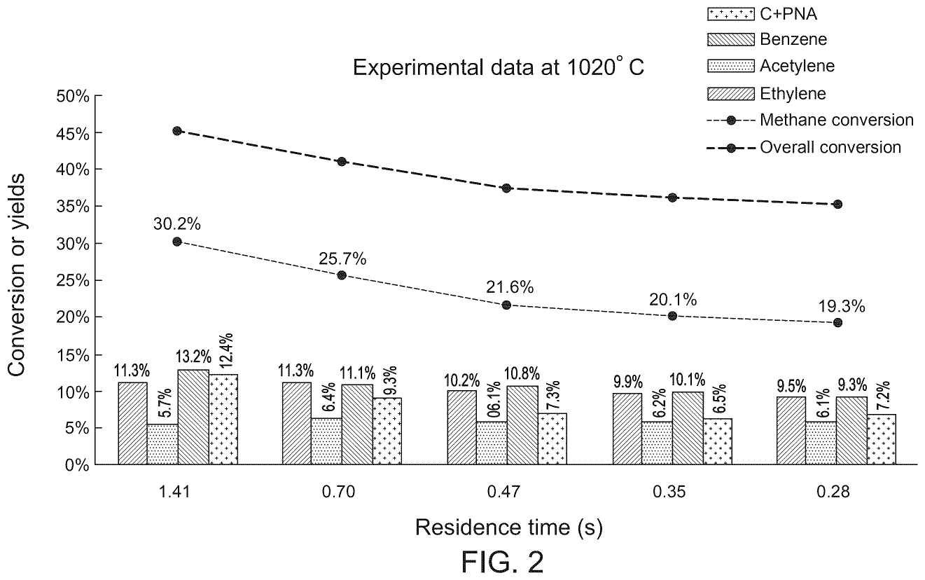

In this example, the reactions used the same feedstock and catalyst as described in Example 1. However, the reaction temperature was increased to 1020° C. and the residence time was varied between 0.28 seconds and 1.41 seconds at ambient pressure. FIG. 2 shows a bar chart illustrating the results. As can be seen in FIG. 2, as the residence time was increased there was a higher overall conversion of methane, ethane and propane in the feed combined, calculated on weight basis, and a greater selectivity for benzene, coke (C) and polynuclear aromatics (PNA). However, the increases in ethylene and acetylene production were minimal, indicating that extending the residence time alone is insufficient to overcome the thermodynamic tendency for higher acetylene production at elevated temperatures.

When comparing the results of Example 1 with the results of Example 2, a reaction conducted at 950° C. with a residence time of 5.46 seconds achieved the same conversion as a reaction at 1020° C. with a residence time of 0.35 seconds. However, the lower temperature (950° C.) condition produced more ethylene and benzene while generating less coke and PNA. In addition, no acetylene was detected at 950° C. with a residence time of 5.46 seconds.

Even when the residence time was extended to 1020° C. to achieve a methane conversion of 30.2% (at 1.41 s residence time), the results offered no advantage compared to the reaction at 950° C. with a residence time of 5.46 seconds. This demonstrates that reaction temperatures at 1020° C. or lower, combined with longer residence times, are more effective at minimizing acetylene formation while maintaining a high conversion as compared to reaction temperatures greater than 1020° C. and shorter residence time which increase acetylene formation and decrease benzene while still maintain a relatively high conversion.

According to an aspect of the present disclosure, a non-oxidative conversion process comprises:
processing a light hydrocarbon feed stream comprising C₁ to C₃ alkanes in a reactor under catalytic cracking conditions comprising a temperature of about 850° C. and up to 1020° C. and a residence time of from about 1 second to about 15 seconds in the presence of a catalytic cracking catalyst, thereby producing a product effluent stream comprising a C₂ to C₁₀ hydrocarbon product and hydrogen,
wherein the product effluent stream contains less than or equal to about 6.5 wt. % of acetylene.

In one or more additional illustrative embodiments, as may be combined with the preceding paragraphs, the product effluent stream contains less than or equal to about 5 wt. % of acetylene.

In one or more additional illustrative embodiments, as may be combined with the preceding paragraphs, the product effluent stream contains less than or equal to about 4 wt. % of acetylene.

In one or more additional illustrative embodiments, as may be combined with the preceding paragraphs, the product effluent stream contains less than or equal to about 3.8 wt. % of acetylene.

In one or more additional illustrative embodiments, as may be combined with the preceding paragraphs, the product effluent stream contains less than or equal to about 2 wt. % of acetylene.

In one or more additional illustrative embodiments, as may be combined with the preceding paragraphs, the product effluent stream contains no amount of acetylene.

In one or more additional illustrative embodiments, as may be combined with the preceding paragraphs, the catalytic cracking conditions comprise a temperature of about 850° C. and up to 1000° C. and a residence time of from about 5.5 second to about 10 seconds.

In one or more additional illustrative embodiments, as may be combined with the preceding paragraphs, the catalytic cracking conditions comprise a temperature of about 920° C. and up to 1000° C. and a residence time of from about 5.5 second to about 10 seconds.

In one or more additional illustrative embodiments, as may be combined with the preceding paragraphs, the catalytic cracking conditions comprise a temperature of about 920° C. and up to 970° C. and a residence time of from about 5.5 second to about 15 seconds.

In one or more additional illustrative embodiments, as may be combined with the preceding paragraphs, the catalytic cracking conditions comprise a temperature of about 920° C. and up to 970° C. and a residence time of from about 7.5 second to about 15 seconds.

In one or more additional illustrative embodiments, as may be combined with the preceding paragraphs, the catalytic cracking conditions comprise a temperature of about 970° C. and up to 1000° C. and a residence time of from about 1 second to about 7.5 seconds.

In one or more additional illustrative embodiments, as may be combined with the preceding paragraphs, the catalytic cracking conditions comprise a temperature of about 850° C. and up to 1000° C. and a residence time of from about 5.5 second to about 10 seconds, and the product effluent stream contains no amount of acetylene.

In one or more additional illustrative embodiments, as may be combined with the preceding paragraphs, the catalytic cracking conditions comprise a temperature of about 920° C. and up to 1000° C. and a residence time of from about 5.5 second to about 10 seconds, and the product effluent stream contains no amount of acetylene.

In one or more additional illustrative embodiments, as may be combined with the preceding paragraphs, the catalytic cracking conditions further comprise a pressure of about 0 atm to about 5 atm.

In one or more additional illustrative embodiments, as may be combined with the preceding paragraphs, the C₂ to C₁₀ hydrocarbon product comprises ethylene and benzene.

In one or more additional illustrative embodiments, as may be combined with the preceding paragraphs, the C₂ to C₁₀ hydrocarbon product comprises from about 1 wt. % to about 25 wt. % ethylene and about 1 wt. % to about 30 wt. % benzene.

In one or more additional illustrative embodiments, as may be combined with the preceding paragraphs, the C₂ to C₁₀ hydrocarbon product comprises from about 5 wt. % to about 15 wt. % ethylene and about 5 wt. % to about 18 wt. % benzene.

In one or more additional illustrative embodiments, as may be combined with the preceding paragraphs, the light hydrocarbon feed stream is a natural gas stream.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present disclosure and are disclosed herein just as if each and every such sub-combination was individually and explicitly disclosed herein.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

What is claimed is:

1. A non-oxidative conversion process comprises:
processing a light hydrocarbon feed stream comprising C₁ to C₃ alkanes in a reactor under catalytic cracking conditions comprising a temperature of about 850° C. and up to 1020° C. and a residence time of from about 1 second to about 15 seconds in the presence of a catalytic cracking catalyst, thereby producing a product effluent stream comprising a C₂ to C₁₀ hydrocarbon product and hydrogen,
wherein the product effluent stream contains less than or equal to about 6.5 wt. % of acetylene.

2. The non-oxidative conversion process according to claim 1, wherein the product effluent stream contains less than or equal to about 5 wt. % of acetylene.

3. The non-oxidative conversion process according to claim 1, wherein the product effluent stream contains less than or equal to about 4 wt. % of acetylene.

4. The non-oxidative conversion process according to claim 1, wherein the product effluent stream contains less than or equal to about 3.8 wt. % of acetylene.

5. The non-oxidative conversion process according to claim 1, wherein the product effluent stream contains less than or equal to about 2 wt. % of acetylene.

6. The non-oxidative conversion process according to claim 1, wherein the product effluent stream contains no amount of acetylene.

7. The non-oxidative conversion process according to claim 1, wherein the catalytic cracking conditions comprise a temperature of about 850° C. and up to 1000° C. and a residence time of from about 5.5 second to about 10 seconds.

8. The non-oxidative conversion process according to claim 1, wherein the catalytic cracking conditions comprise a temperature of about 920° C. and up to 1000° C. and a residence time of from about 5.5 second to about 10 seconds.

9. The non-oxidative conversion process according to claim 1, wherein the catalytic cracking conditions comprise a temperature of about 920° C. and up to 970° C. and a residence time of from about 5.5 second to about 15 seconds.

10. The non-oxidative conversion process according to claim 1, wherein the catalytic cracking conditions comprise a temperature of about 920° C. and up to 970° C. and a residence time of from about 7.5 second to about 15 seconds.

11. The non-oxidative conversion process according to claim 1, wherein the catalytic cracking conditions comprise a temperature of about 970° C. and up to 1000° C. and a residence time of from about 1 second to about 7.5 seconds.

12. The non-oxidative conversion process according to claim 1, wherein the catalytic cracking conditions comprise a temperature of about 850° C. and up to 1000° C. and a residence time of from about 5.5 second to about 10 seconds, and the product effluent stream contains no amount of acetylene.

13. The non-oxidative conversion process according to claim 1, wherein the catalytic cracking conditions comprise a temperature of about 920° C. and up to 1000° C. and a residence time of from about 5.5 second to about 10 seconds, and the product effluent stream contains no amount of acetylene.

14. The non-oxidative conversion process according to claim 1, wherein the catalytic cracking conditions further comprise a pressure of about 0 atm to about 5 atm.

15. The non-oxidative conversion process according to claim 1, wherein the C₂ to C₁₀ hydrocarbon product comprises ethylene and benzene.

16. The non-oxidative conversion process according to claim 1, wherein the C₂ to C₁₀ hydrocarbon product comprises from about 1 wt. % to about 25 wt. % ethylene and about 1 wt. % to about 30 wt. % benzene.

17. The non-oxidative conversion process according to claim 1, wherein the C₂ to C₁₀ hydrocarbon product comprises from about 5 wt. % to about 15 wt. % ethylene and about 5 wt. % to about 18 wt. % benzene.

18. The non-oxidative conversion process according to claim 1, wherein the light hydrocarbon feed stream is a natural gas stream.
